(19) United States
(12) Patent Application Publication
(10) Pub. No.: US 2010/0030338 A1
(43) Pub. Date: Feb. 4, 2010

(54) PROSTHESIS FOR NUCLEUS OR INTER-VERTEBRAL DISC REPLACEMENT

(75) Inventor: Lionel Simon, Grigny (FR)

Correspondence Address: HESLIN ROTHENBERG FARLEY & MESITI PC, 5 COLUMBIA CIRCLE, ALBANY, NY, 12203, US

(73) Assignee: SPINEWAY, Bron (FR)

(21) Appl. No.: 12/519,298

(22) PCT Filed: Dec. 13, 2007
(86) PCT No.: PCT/FR2007/002060

§ 371 Date: Aug. 4, 2009

(30) Foreign Application Priority Data
Dec. 13, 2006 (FR) 0610852
Apr. 12, 2007 (FR) 0702659

(51) Int. Cl. A61F 2/44

(52) U.S. Cl. 623/17.16

(57) ABSTRACT

The invention relates to a nucleus or inter-vertebral disc prosthesis that comprises at least two plates capable of movement relative to each other through at least one articulation interface defined by a female surface and a male surface each having an essentially spherical shape and essentially equal diameters. The two articulation surfaces are respectively intersected by surfaces that define between them a relative mobility space for the plates when the articulation surfaces are coapted; the surface intersecting the female surface being contained in the volume defined by an equatorial plane of said sphere except for said equatorial plane itself, the surface intersecting the male surface being planar or convex.

### FIELD OF THE INVENTION

The present invention relates to a prosthesis for completely replacing a damaged inter-vertebral disc of the human vertebral column, or for partially replacing it, by substituting only for its “nucleus pulposus”. Its field of application is therefore orthopaedic surgery.

In the vertebral column, each vertebra is separated from the adjacent vertebrae by a fibro-cartilaginous disc, called inter-vertebral disc. These discs have a support, articulation and damping role between each vertebra.

The degeneration of these discs harms their functions and can cause pain or disabling functional limitations.

One of the techniques used to treat patients suffering from such disorders is to resort to surgery to replace the complete degenerative disc, or only its “nucleus pulposus”, by a prosthesis restoring the functions of the excised organ as closely as possible.

### PRIOR ART

Various articular prostheses for inter-vertebral discs have been proposed to date, designed to meet a number of fundamental requirements.

The first of these requirements, described for example in documents U.S. Pat. No. 5,246,458 and WO9113598, is to allow three rotations:
the first about an axis substantially equivalent to the axis of the vertebral column (rotation),
the second about an axis perpendicular to the sagittal plane (flexion-extension),
and the third about an axis perpendicular to the frontal plane (tilting).
   Another requirement is to reduce the contact stresses in order to preserve the articular surfaces. This is done by favouring a surface support and hence a permanent congruence of the two articular surfaces during their relative movement, and also the largest possible articular surfaces.

The known prior art documents, and for example document WO9113598, already mentioned, or document FR 2 865 629, omit this parameter, thereby necessitating the use of a hard-on-hard bearing couple, failing which, the functioning of the prosthesis is prevented for relatively low wear of the surfaces.

The simultaneous answer to these two requirements implies the provision of at least one interface called articular, consisting in concrete form of two matching surfaces of essentially spherical shape, rigid, having virtually equal radii, and hence the provision of an implant comprising at least two elements.

The final fundamental requirement of an articular prosthesis is to enjoy sufficient stability to prevent its luxation.

Other requirements, less fundamental from the kinematic standpoint, are nevertheless essential from the anatomical, operative standpoint, and in terms of manufacture.

In fact, as observed previously, the space available to lodge the implant is limited.

Furthermore, at every moment of the movement, two vertebrae in relative movement define an instantaneous centre of rotation. The anatomical instantaneous centre of rotation between two vertebrae, even if slightly mobile, has a known position, below the inter-vertebral space between the centre and the posterior third of the vertebral plates. Compliance with this position avoids the creation of non-anatomical movements, which are liable to damage the posterior articulations of the vertebral column.

The answer to this requirement may assume two different forms. The first consists in designing an implant of which the centre of rotation is fixed and located in the appropriate space (see for example US2006229725 and WO0101893). The second answer to this requirement consists in designing an implant of which the centre of rotation is mobile, leaving the posterior articulations free to restrict the relative movement of the two vertebrae adjacent to the implant. The latter alternative implies the provision of a second articular interface, and hence the provision of an implant comprising at least three elements (see for example EP1532950 or FR2865629).

Furthermore, it has been found that the penetration of the scar tissues into the spaces left free in the volume of the prosthesis could cause it to be blocked, and eventually its fusion in a bony massif. Document U.S. Pat. No. 5,683,465 addresses this problem.

Similarly, the provision of spherical articular surfaces systematically resorts to the turning technology, which has the drawback of generating a surface defect at the turning axis. This defect is generally located at the centre of the articular surface and can therefore cause substantial wear of the implant. It is therefore important to control this defect, or to eliminate the contact at this location.

Finally, it may be observed that high stability between the components and the prosthesis is useful in case of extraction, because it then suffices to secure only one of the components in order to extract the whole.

The solutions proposed by the prior art are unable to meet all of these various requirements.

Thus document EP1532950 describes an implant which leaves a large peripheral space free, which is favourable to bone fusion. This space cannot be filled by the core because the radii of the articular surfaces, which are necessarily lower than the height of the implant, limit its diameter in a plane perpendicular to its axis.

Document WO2005007040 describes an implant provided with an element for joining the two plates constituting it, the said element necessarily passing through the articular surface. Thus, it needlessly increases the height of the implant.

Documents FR2856587 and U.S. Pat. No. 5,683,465 describe an implant in which the centre of rotation is fixed and does not comply with the anatomical centre of rotation. Moreover, the radius of curvature of the articular surfaces is necessarily very short, to allow suitable displacement, thereby freeing a large peripheral space, which is favourable to bone fusion, and therefore contrary to the desired result.

Document U.S. Pat. No. 5,895,428 describes an implant in which the spherical articular surfaces are surmounted by a screw having the function of maintaining all the various components. This configuration needlessly increases the height of the implant. Moreover, as it is presented, the implant does not comply with the anatomical centre of rotation, and further comprises peripheral spaces which are favourable to bone fusion.

### SUMMARY OF THE INVENTION

It is the object of the device according to the invention to overcome the various deficiencies of the implants proposed by the prior art, by simultaneously meeting all the requirements discussed and by providing improvements both in terms of manufacture and use, that is:
allow the three rotations discussed in the introduction,
ensure a permanent congruence during the relative movement of the articular surfaces,
ensure high stability to prevent luxation, and facilitate extraction if necessary,
comply with the anatomical centre of rotation by causing it to coincide with the prosthetic centre of rotation, and even allowing some mobility of the prosthetic centre of rotation,
complying with the inter-vertebral space available by limiting the height of the implant,
allowing a maximum increase of the articular surfaces and a functioning that tolerates the wear and hence the use of soft materials such as polyethylene,
minimize the free spaces that are favourable to fusion,
allow for simple manufacture by eliminating the problem of wear associated with the surface defect resulting from turning,
allow extremely simple use based on a minimum of two components.

For this purpose, the device according to the invention consists of a prosthesis for nucleus or inter-vertebral disc replacement comprising at least two plates, anchored respectively in the adjacent vertebrae via anchoring surfaces, the said plates being mobile with regard to one another via at least one interface defined by two so-called articular surfaces, respectively a female surface and a male surface, the said articular surfaces having an essentially spherical shape and substantially equal curvature radii.

According to the invention, the two articular surfaces are intersected respectively by surfaces which, when the said articular surfaces are coapted, together define a space of relative mobility of the plates, the surface intersecting the female articular surface being contained in the volume defined, on the one hand, by the theoretical sphere of the said female articular surface to the exclusion of a point which defines a pole, and, on the other hand, by an equatorial plane of the said sphere to the exclusion of the said equatorial plane itself, the surface intersecting the male articular surface being planar or convex and being contained in a volume bounded, on the one hand, by the theoretical sphere of the said male articular surface, and, on the other hand, by the equatorial plane to the exclusion thereof

Advantageously, the surfaces that intersect the articular surfaces are located in the neighbourhood of the pole of the interface, the said pole being defined with regard to an axis parallel to the approximate axis of revolution of the natural inter-vertebral disc.

According to an alternative embodiment of the invention, at least one of the surfaces that intersect the articular surfaces is planar.

According to an alternative embodiment of the invention, both surfaces that intersect the articular surfaces are planar.

According to one version of the invention, the mobility space arranged is filled at least partially by a deformable body.

According to an alternative embodiment, the implant comprises a third element acting as a mobile core, defining two interfaces with the plates, on the one hand the interface between the core and one of the two plates, and on the other hand, the interface between the core and the other plate.

According to an alternative embodiment, the two interfaces are essentially spherical, oriented in the same direction, and one of the two interfaces is included in the sphere defined by the other.

According to an alternative embodiment, one of the two interfaces is planar, the other is concave.

Thus, the articular prosthesis according to the invention serves to provide the best compromise between the following requirements: position of the centre or centres of rotation of one or both articular interfaces, area of the contact surface to reduce wear, thickness of the implant, mobility required by the implant, inclination of the interfaces to prevent the expulsion of the core if any or the luxation of the prosthesis, and filling of the free spaces, which are favourable to fusion.

### BRIEF DESCRIPTION OF THE FIGURES

Various other features will appear better from the description that follows, in conjunction with the appended drawings which show, by way of example, embodiments of the object of the invention. These examples are non-limiting, and combinations are possible.

The various figures show cross sections of an implant according to the invention, of which the head side is at the top and the sacrum side at the bottom, and which could be axisymmetrical.

FIG. 1 shows a cross section of a healthy natural disc between two vertebrae, the prosthesis implant location.

FIGS. 2 to 8 show implants having only one articular interface. These implants can nevertheless be provided in a number of components higher than two, but fixedly joined to finally result in only two solids moving with regard to one another.

FIGS. 9 to 13 show implants comprising two articular surfaces.

FIG. 14 is a schematic diagram.

### DESCRIPTION OF EMBODIMENTS OF THE INVENTION

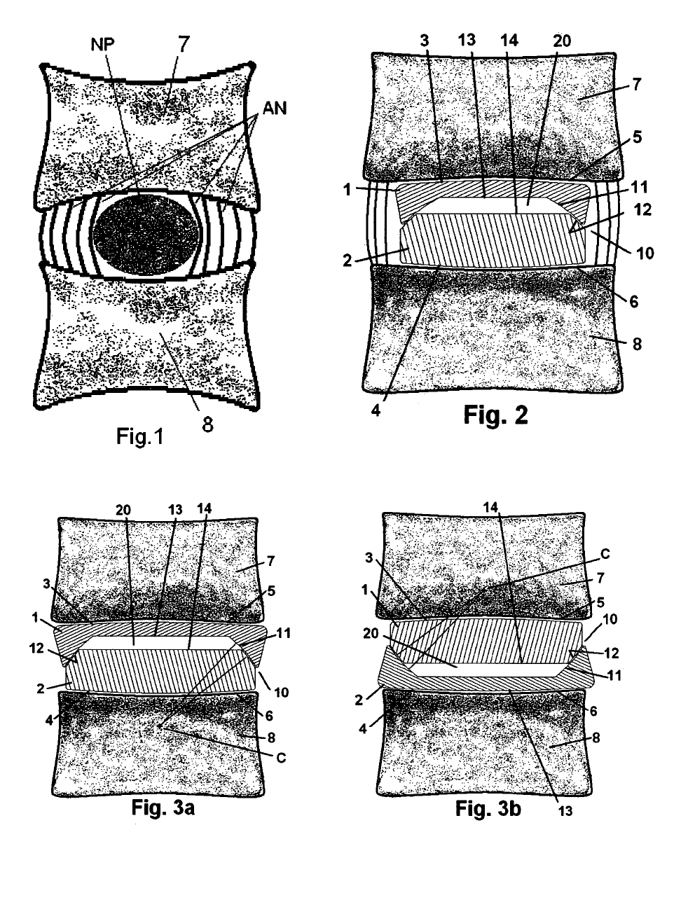

FIG. 1 shows a healthy natural disc between two vertebrae (7) and (8), comprising its nucleus pulposus (NP) and its annulus (AN).

FIG. 2 shows a nucleus replacement implant according to the invention. Its size is reduced with regard to a complete prosthesis, in order to preserve part of the annulus.

FIG. 3a shows a complete disc prosthesis according to the invention, in place between the vertebrae (7) and (8). In this particular embodiment, the surfaces that intersect the articular surfaces are planar and approximately located in the neighbourhood of the pole of the articulation, and the instantaneous centre of rotation (C) is located below the inter-vertebral space.

FIG. 3b shows a complete disc prosthesis according to the invention, virtually identical to that of FIG. 3a, but for which it was decided to position the instantaneous centre of rotation (C) above the inter-vertebral space. This configuration does not comply with the anatomical centre of rotation and may thus cause pain to the patient, but is technically feasible.

According to the embodiment shown in FIG. 3a, the plates (1) and (2) can each be made in a single block of chromium-cobalt alloy, which is particularly advantageous for its tribological properties, and be suitable for friction against one another.

According to the same illustration, one of the plates (1) and (2) can be made in two parts, one of chromium-cobalt, the other of polyethylene, the two parts then being joined fixedly to form the said plate, the other plate being made from a block of chromium-cobalt, for example, the friction couple between the chromium-cobalt and the polyethylene being very commonly used in vertebra, hip or knee replacement.

Furthermore, the anchoring surfaces (3) and (4) of the plates (1) and (2) to the adjacent vertebral plates (5) and (6) may be convex in order to adapt to their anatomy as closely as possible, and covered with a coating that promotes recolonization by the bone, a coating of pure titanium applied by plasma spraying is often employed. According to this embodiment of the invention, the polyethylene part is positioned fixedly in the lower plate (2) whereof it becomes an integral part, the plate itself being anchored to the lower vertebra not shown, and the upper plate (1), anchored in the upper vertebra not shown is mobile with regard to the plate (2). The discal prosthesis thus provided allows the restoration of the discal space and the mobility between the vertebrae.

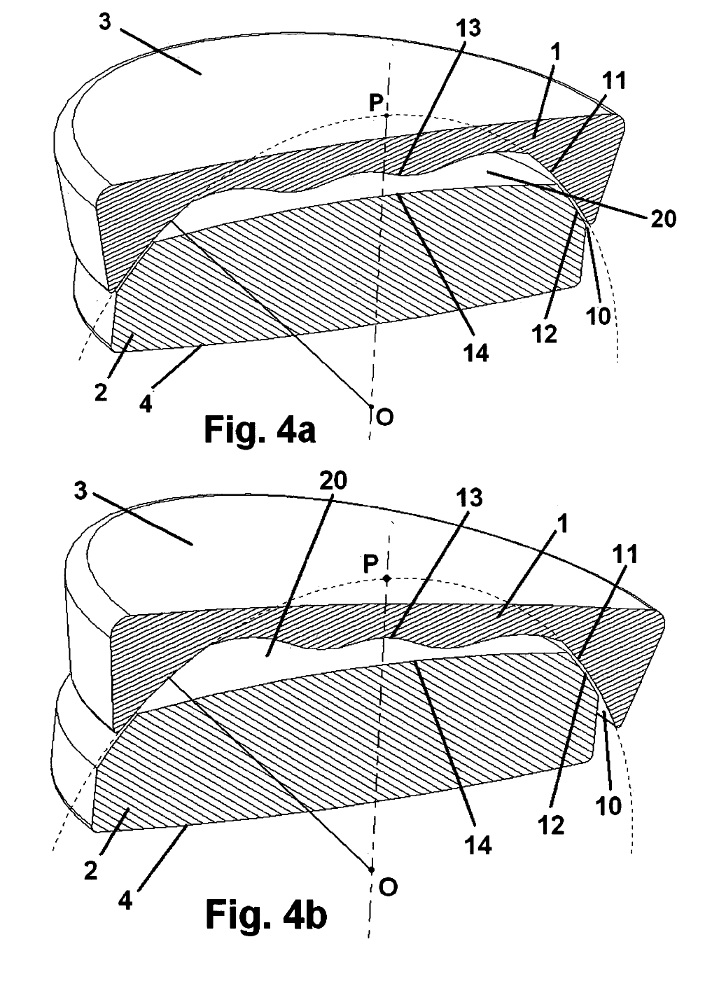

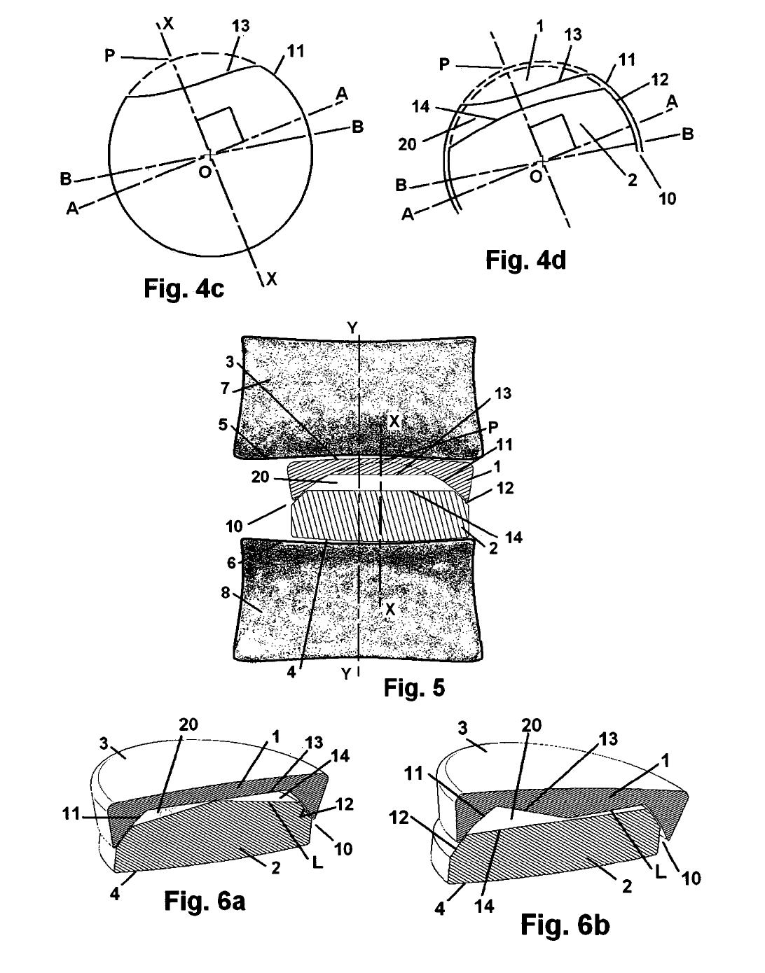

FIGS. 4a and 4b show the general principle of the invention, that is two plates (1) and (2) respectively comprising spherical articular surfaces (11) and (12) having approximately equal radii. Each of these articular surfaces (11) and (12) is intersected by a surface (13) and (14).

This embodiment, according to the invention, represents a surface (13) of any type, and a convex surface (14). This figure is intended to demonstrate certain advantages that the invention simultaneously provides.

Firstly, no free space favourable to intrusion of scar tissue subsists, regardless of the relative position of the plates (1) and (2).

Moreover, the gain in height of the prosthesis is demonstrated by the outline of the theoretical sphere defined by the female articular surface (11) which culminates above the volume of the plate (1).

Furthermore, the mobility space (20) thereby defined allows the movement illustrated in FIG. 4b.

Moreover, the prosthetic centre of rotation (O) complies with the anatomical centre.

Furthermore, the inclination of the articular surfaces is greater than in the case of a polar contact, thereby providing greater stability.

Moreover, the pole (P) of the articulation, or end of the female theoretical sphere, which generally has a turning defect, is not a contact point, thereby facilitating the construction of the implant because it is no longer necessary to address this problem.

Finally, the implant only comprises two components, which facilitates its handling and therefore its use.

FIG. 4c is intended to explain how the designation, on a sphere, for example that defined by the articular surface (11), of a particular point as a pole (P), naturally requires the definition of:
a single axis (XX) of revolution of the sphere joining the pole (P) and the centre (O) of the sphere;
a single equatorial plane (AA), therefore passing by definition through the centre (O) of the sphere, perpendicular to the axis (XX).

An equatorial plane (BB) can also be defined, passing by definition through the centre (O) of the sphere, and having an orientation close to that of the plane (AA).

It is thereby possible to define any surface (13) of intersection of the articular surface (11) contained in a volume defined, on the one hand, by the theoretical sphere of the said surface (11) to the exclusion of a selected polar point (P) and, on the other hand, by an equatorial plane (BB) approximately perpendicular to the line joining the pole (P) and the centre (O) to the exclusion of the said equatorial plane (BB) itself

FIG. 4d is intended to explain the possible place of presence of the surface (14) of intersection of the male articular surface (12), convex here and contained in a volume bounded on the one hand by the theoretical sphere of the said surface (12), and on the other hand, by the equatorial plane (BB) defined previously to the exclusion thereof.

FIG. 5 is intended to illustrate the choice of a pole (P) defined by an axis (XX) parallel to the approximate axis (YY) of revolution of the inter-vertebral disc. In this example, the two intersection surfaces (13) and (14) are planar.

FIGS. 6a and 6b show the possibility offered by the invention of providing an internal angular stop system.

In FIG. 6a, the intersection surface (13) is planar, and the intersection surface (14) is essentially conical and adapted in that it allows a contact along a line (L) when the plate (1) reaches the maximum displacement selected.

FIG. 6b shows the plate (1) inclined and close to the stop along a line (L) between the essentially conical surface (13), and the planar surface (14).

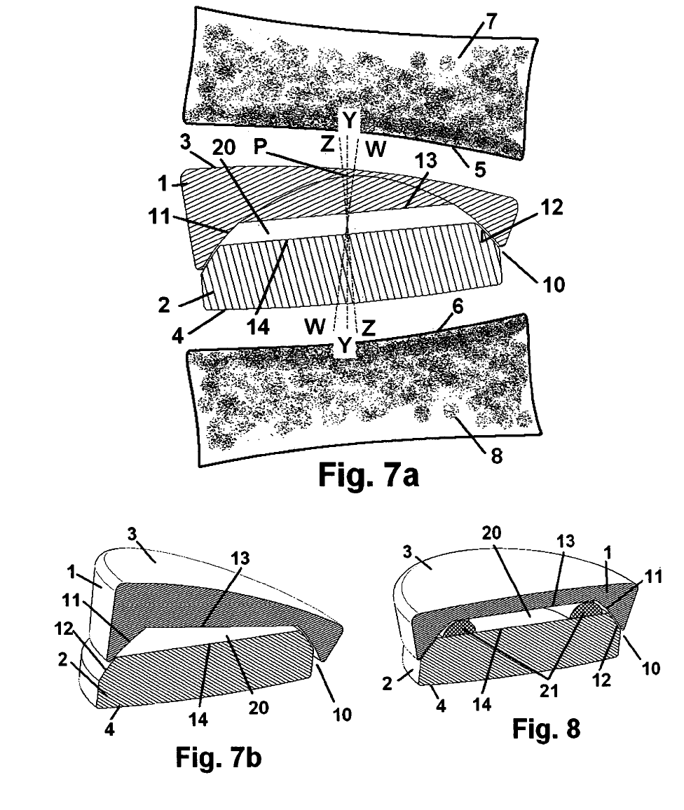

FIG. 7a shows a particular embodiment of the invention for which it is considered that the axis (YY) of revolution of the inter-vertebral disc is an approximation of the axes of revolution (ZZ) and (WW) of each of the vertebral plates (5) and (6). Thus, the pole (P) is approximately defined.

FIG. 7b shows the same implant as FIG. 7a, at the displacement limit.

FIG. 8 shows an embodiment of the implant in which part of the mobility space (20) if filled by a deformable body (21), for example of silicone or polyurethane, in order, for example, to provide a progressive stop system which allows a more uniform distribution of the mobility between the prosthetized stage and the other natural stages of the vertebral column. In fact, a natural disc offers some resistance to the relative movement of two vertebrae, whereas a prosthesis like those that exist today virtually offers no resistance. This imbalance may at least temporarily lead to a hypermobility of the prosthesized level, causing a local deformation of the vertebral column, for example a cervical kyphosis, or an exaggerated stress of the posterior articulations on the one hand, and in exchange a reduction of the mobility of the healthy adjacent stages which may lead to their rigidification.

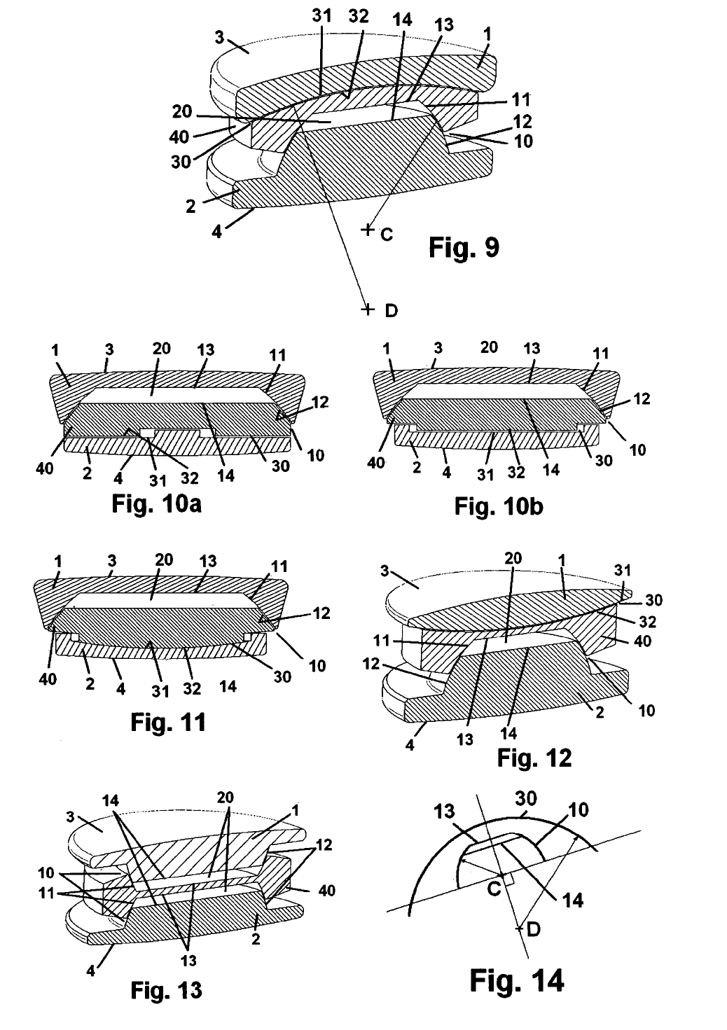

FIG. 9 is intended to illustrate an implant comprising two plates (1) and (2) and a core (40). This embodiment has the special feature of having two articular interfaces (10) and (30) with the same orientation, one (10) called internal, being inside the other (30), called external. In particular, the centre (C) of the internal interface (10) is located between the external interface (30) and its own centre (D). The advantage of this particular arrangement is to define a stable position of the plate (1) with regard to the core (40). In fact, if the plate (1) moves from this position with regard to the core (40), the distance between the plate (1) and the plate (2) increases, thereby separating the vertebrae (7) and (8) not shown, from one another. The anatomical forces exerted on the implant therefore have the consequence of returning the plate (1) to its stable position with regard to the core (40), so that the height of the implant is again minimized.

The following advantages resulting from this embodiment of the invention can be identified:
the rotation and translational movements of a vertebra with regard to another are uncoupled owing to the difference in curvature radii, the interface (10) essentially providing the rotations, and the interface (30) the translations. This effect is advantageous for avoiding an excessive loading of the posterior articulations if the implant is poorly positioned;
the self-stability of the interface (30) described above prevents an excessive loading of the posterior articulations of the vertebral column which, in this case, no longer have to withstand alone the burden of containing the translation;
the gain in height at the interface (10), serves to reduce the size of the implant;
the gain in stability of the interface (10) and the self-stability at the interface (30) described above serve to avoid the expulsion of the core (40).

FIG. 10a is intended to illustrate an implant comprising two plates (1) and (2) and a core (40). This embodiment has the special feature of having a planar articular interface (30) and an internal device limiting the translational displacement.

FIG. 10b is intended to illustrate a particular embodiment having a planar articular interface (30) and an external device limiting the translational displacement.

FIG. 11 is intended to illustrate a particular embodiment having an essentially spherical articular interface (30) and an external device limiting its displacement.

FIGS. 12 and 13 show other embodiments of the interfaces (10) and (30).

FIG. 14 shows a schematic diagram illustrating the possible place of presence of the interface (10) whereof the centre (C) is located on a radius of the interface (30) and the interface (10) is itself located between the interface (30) and a plane passing through its own centre (C) and approximately perpendicular to the line joining the centres (C) of the interface (10) and (D) of the interface (30).

The invention is not limited to the various examples described and shown because various modifications can be made thereto without going beyond its scope.

1. Prosthesis for a nucleus or inter-vertebral disc replacement comprising at least two plates, cooperating respectively with one and an other of two vertebral plates of adjacent vertebrae via respective anchoring surfaces, the plates being mobile with regard to one another via at least one interface defined by two articular surfaces comprising a female surface and a male surface, the articular surfaces having an essentially spherical shape and substantially equal curvature radii, wherein:
the articular surfaces are intersected respectively by surfaces which, when the said articular surfaces and are coapted, together define a space of relative mobility of the two plates in the following three rotations in space, a first, rotation, about an axis substantially equivalent to an axis of a vertebral column, a second, flexion-extension, about an axis perpendicular to a sagittal plane, and a third, tilting, about an axis perpendicular to a frontal plane;
the surface intersecting the female surface is contained in a volume defined by a theoretical sphere of the female surface to the exclusion of a polar point, and by an equatorial plane to the exclusion of the said equatorial plane itself; and
the surface intersecting the male surface is planar or convex and is contained in a volume bounded by a theoretical sphere of the male surface, and by the equatorial plane to the exclusion of the equatorial plane.

2. Prosthesis for nucleus or inter-vertebral disc replacement according to claim 1, wherein the polar point is defined by an axis parallel to an approximate axis of revolution of the inter-vertebral disc.

3. Prosthesis for nucleus or inter-vertebral disc replacement according to claim 1, wherein at least one of the intersection surfaces is planar.

4. Prosthesis for nucleus or inter-vertebral disc replacement according to claim 1, wherein both intersections surfaces are planar.

5. Prosthesis for nucleus or inter-vertebral disc replacement according to claim 1, wherein space between the intersection surfaces is filled at least partially by a deformable body.

6. Prosthesis for nucleus or inter-vertebral disc replacement according to claim 1, further comprising a third element acting as a mobile core, defining two interfaces with the plates:
a first interface between the core and one of the two plates and,
a second interface between said core and the other plate, defined by two articular surfaces movable with regard to one another.

7. Prosthesis for nucleus or inter-vertebral disc replacement according to claim 6, wherein:
the second interface is essentially spherical,
a centre of the first interface is located on a segment joining the second interface and a centre of the second interface,
the first interface is included in a volume bounded by the second interface, and a plane passing through the centre of the first interface and perpendicular to a line passing through the centre of the first interface and the centre of the second interface.

8. Prosthesis for nucleus or inter-vertebral disc replacement according to claim 6, wherein:
the second interface is planar, and
the first interface is located between a plane of the second interface, and the plane parallel to the second interface passing through the centre of the first interface.
